(19) United States
(12) Patent Application Publication
(10) Pub. No.: US 2006/0254909 A1
(43) Pub. Date: Nov. 16, 2006

(54) Electrode and sensor using same

(75) Inventors: Izumi Kubo, Tokyo (JP); Nobuyoshi Maehara, Tokyo (JP)

Correspondence Address: BIRCH STEWART KOLASCH & BIRCH, PO BOX 747, FALLS CHURCH, VA, 22040-0747, US

(73) Assignee: TAMA-TLO Corporation, Tokyo (JP)

(21) Appl. No.: 10/549,479

(22) PCT Filed: Mar. 16, 2004
(86) PCT No.: PCT/JP04/03467

§ 371 Date: Jun. 28, 2006

(30) Foreign Application Priority Data
Mar. 18, 2003 (JP) 2003-074543

(51) Int. Cl. G01N 33/487

(52) U.S. Cl. 204/403.01

(57) ABSTRACT

To prevent an electrode improvable to measurement accuracy by using a novel self-assembled monolayer suppressing an influence of the interference substance in measuring an object to be measured, and a sensor using the same. The sensor has a modified electrode 12 and a counter electrode. The modified electrode 12 has a gold electrode 16 as an electrode base, a self-assembled monolayer 18 covering the gold electrode 16 and made of carboxyalkanethiol expressed by a chemical structural formula of HS(CH₂)ₙCOOH (n=5 to 9), and an enzyme 19 immobilized on the self-assembled monolayer 18. When measuring fructose, FDH may be used as the enzyme 19. The self-assembled monolayer 18 allows Co(phen)₃²⁺ as a mediator to pass through selectivity when being formed of 7-carbxy-1-heptanethiol.

### TECHNICAL FIELD

The present invention relates to a sensor measuring an object to be measured by utilizing an electrochemical reaction, and an electrode used in the same.

### BACKGROUND ART

There is known a variety of chemical sensors or biosensors which measure an object to be measured (measuring object) in a sample by utilizing an electrochemical reaction. As one type of these sensors, there is known a sensor in which a substance for reacting the object and an electrode is immobilized to an electrode by a self-assembled monolayer.

The sample measured by the sensor may include, as impurities other than the object, an interference substance for interfering in the electrochemical reaction of the object and the electrode. And, it has been pointed out that the interference substance affects a measured result of the object. As the interference substance affecting the measured result, for example, ascorbic acid and uric acid are known.

For example, if ascorbic acid is included in the sample when measuring fructose (fruit sugar), both of a current caused by an oxidation reaction of the fructose and a current caused by an oxidation reaction of the ascorbic acid may flow to an electrode. Consequently, an accurate measurement of concentration of fructose is difficult.

On the other hand, in order to prevent an electrochemical reaction caused by ascorbic acid, uric acid, or other impurities which exist in biosample relatively in large quantities, a technology employing 10-carboxy-1-decanethiol as the self-assembled monolayer is disclosed (referred to Hiroaki SHINOHARA, “High Selectivity Sensing of Organism Important Substance by Designing Electrode Interface Molecular Accumulation Film”, Construction of Structure Controlling Function Interface and Electrode Reaction, Result report in Heisei 10 year, 1999, p. 145 to 146).

### DISCLOSURE OF THE INVENTION

An object of the present invention is to provide an electrode improvable to measurement accuracy by using a novel self-assembled monolayer which suppresses an influence of the interference substance in measuring an object to be measured, and a sensor using the same.

To achieve the object, according to the present invention, there is provided an electrode having: an electrode base, and a self-assembled monolayer expressed by a chemical structural formula of HS(CH₂)ₙCOOH (n=5 to 9) and covering the electrode base.

To achieve the object, according to the present invention, there is also provided a sensor having: a vessel receiving a sample solution in which an object to be measured dissolves, and a modified electrode and a counter electrode to be dipped into the sample solution. The modified electrode includes an electrode base, and a self-assembled monolayer expressed by a chemical structural formula of HS(CH₂)ₙCOOH (n=5 to 9) and covering the electrode base.

### BRIEF DESCRIPTION OF THE DRAWINGS

FIG. 1 is a view of a configuration of a sensor according to the present embodiment.

FIG. 2A is a plan view of a configuration of a modified electrode according to the present embodiment, and FIG. 2B is a cross-sectional view along section I-I of FIG. 2A.

FIG. 3 is a view of a configuration of a fructose sensor.

FIG. 4 is a cross-sectional view of the modified electrode used to a verification test of selectivity of a self-assembled monolayer.

FIG. 5 is a cyclic voltammogram for ascorbic acid given by the verification test of selectivity of the self-assembled monolayer.

FIG. 6 is a cyclic voltammogram for Co(phen)₃²⁺ given by the verification test of selectivity of the self-assembled monolayer.

FIG. 7 is a view of a construction of a glucose sensor.

FIG. 8 is a view showing a measured result of a glucose concentration by using the glucose sensor in which Co(phen)₃²⁺ is used as a mediator.

FIG. 9 is a view of a construction of a catecholamine sensor.

FIG. 10 is a view showing a measured result of a reaction of dopamine and ascorbic acid by using the catecholamine sensor.

FIG. 11 is a view showing a calibration curve of dopamine by using the catecholamine sensor.

FIG. 12 is a view showing a relationship between alkyl chain length of lipid included in the self-assembled monolayer and dopamine selectivity.

FIG. 13 is a plan view of a sensor module according to a modification of an embodiment of the present invention.

### BEST MODE FOR CARRYING OUT THE INVENTION

Below, embodiments of the present invention will be described with reference to the attached drawings.

(Configuration of Apparatus)

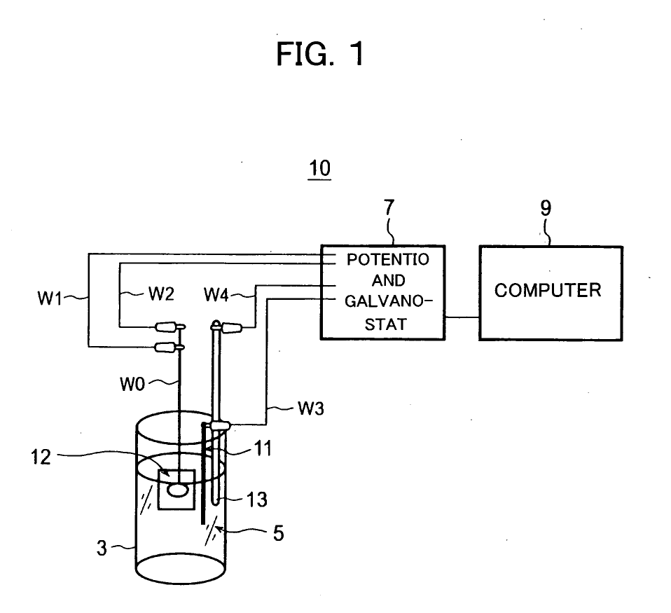

FIG. 1 is a view of a configuration of an example of a sensor according to the present invention.

A sensor 10 shown in FIG. 1 has a vessel (vial) 3 receiving a sample solution 5 in which an object to be measured (measuring object) its concentration dissolves, a counter electrode 11 made of platinum (Pt) wire, a modified electrode (working electrode) 12, a reference electrode 13 made of silver and silver chloride (Ag/AgCl), a potentio and galvanostat apparatus 7 as a voltage applying part, and a computer 9 as a calculation part.

The counter electrode 11, the modified electrode 12, and the reference electrode 13 are, for example, fixed on a cover not shown in the drawings and dipped into a sample solution 5 in the vessel 3. As the counter electrode 11, for example, silver and silver chloride (Ag/AgCl) electrode can be used other than platinum wire. Also, as the reference electrode 13, for example, a saturated calomel electrode can be used other than Ag/AgCl electrode.

The potentio and galvanostat apparatus 7 is connected via wires W0, W1 and W2 to the modified electrode 12, connected via a wire W3 to the counter electrode 11, and connected via a wire 4 to the reference electrode 13.

The potentio and galvanostat apparatus 7 is an apparatus having both functions of a potentiostat for controlling voltage of the modified electrode 12 within a certain range and a galvanostat for detecting a current flowing between the modified electrode 12 and the counter electrode 11, and used to an electrochemical measurement in general. A detected current value is send to the computer 9.

The potentio and galvanostat apparatus 7 applies a predetermined voltage to the modified electrode 12 to cause an electrode reaction in the electrode. If a reaction in which an electron is given to substance in a solution occurs in the modified electrode 12, the reverse reaction (the electron is delivered from the substance in the solution) may be caused in the counter electrode 11 serving as an another electrode to flow a current in a circuit, which allows a measurement of the electrochemical reaction.

When applying the predetermined voltage to the modified electrode 12, in the present embodiment, the potentio and galvanostat apparatus 7 has supplied a predetermined voltage to the reference electrode 13 which is different from the modified electrode 13 and the counter electrode 11. Then, by using the applied voltage to the reference electrode 13 as a reference, the apparatus 7 applies the predetermined voltage to the modified electrode 12.

The computer 9 decides a concentration of the measuring object from a measured current value of the sample solution 5 in the vessel 3, for example, based on a calibration curve which indicates a relationship between a concentration of the measuring object and the current and which is stored on a memory in advance. The computer 9 outputs the calculated concentration of the measuring object, for example, to a display unit and the display unit displays the calculated concentration.

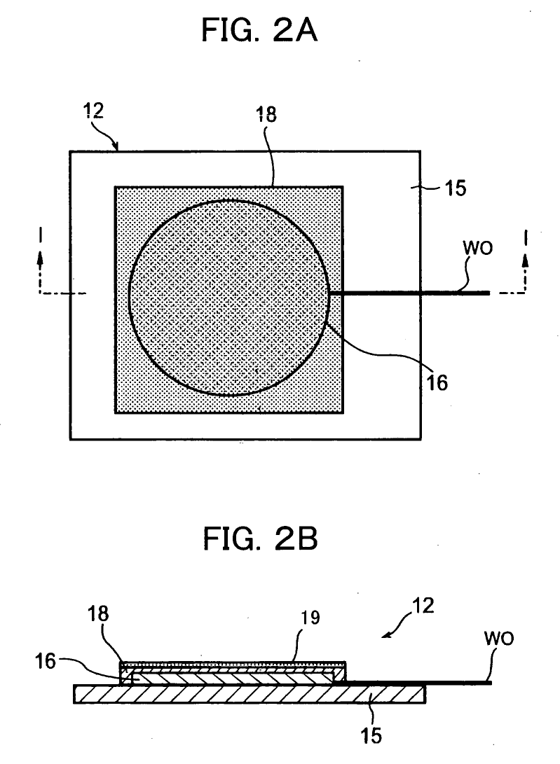

FIGS. 2A and 2B are views of a configuration of an embodiment of the modified electrode 12 according to the present embodiment. FIG. 2A is a plan view of the modified electrode 12 and FIG. 2B is a cross-sectional view along section I-I of FIG. 2A.

The modified electrode 12 has a gold electrode (electrode base) 16 fixed on a substrate 15 of glass or plastic, a self-assembled monolayer 18 covering a surface of the gold electrode 16, and an enzyme 19 immobilized on the self-assembled monolayer 18. The gold electrode 16 is connected to the wire W0 for applying voltage and for measuring a current.

As the measurement object, for example, fructose, glucose, catecholamine, quinone, serotonin, hydrophobic amino acid, or other chemical or biological materials are mentioned.

The self-assembled monolayer 18 is configured by carboxyalkanethiol expressed by a chemical structural formula of HS(CH₂)ₙCOOH (n=5 to 9). For example, it is configured by one of 5-carboxy-1-pentantiol of n=5,6-carboxy-1-hexantiol of n=6,7-carboxy-1-heptantiol of n=7,8-carboxy-1-octantiol of n=8, and 9-carboxy-1-nonanetiol of n=9.

The thiol compounds react a gold surface to form a gold-systeamine (Au—S) bonding. Among them, by using an alkanthiol compound, the Au—S bonding and additionally Van der Waals interaction between the alkyl chains function. Consequently, it becomes possible to form a monolayer having a high orientation and free from defects. Such monolayer is called as a self-assembled monolayer. A modification of the gold electrode 16 by the self-assembled monolayer 18 can be performed only by dipping the gold electrode 16 into a solution of thiol compounds.

The self-assembled monolayer 18 according to the present embodiment repels ascorbic acid, uric acid or other interference substance, which is water solubility and activated in electrochemical, by its carboxyl group to hinder an arrival of the substance to the gold electrode 16.

The enzyme 19 is selected, for example, based on the measuring object. If the measuring object is fructose, the enzyme 19 may be used with, for example, fructose dehydrogenase (FDH). If the measuring object is glucose, the enzyme 19 may be used with, for example, glucose oxidase (GOD) serving as an oxidase of glucose.

In the present embodiment, the oxidation reaction of the measuring object by using the enzyme 19 is mediated by a mediator (a mediation part or electrochemical activator), and converted to an electric signal in the gold electrode 16. By detecting the electric signal, an existence of the measuring object can be recognized.

The mediator is preferably hydrophobicity. As such mediator, for example, phenanthroline cobalt (II) complex [Co(phen)₃²⁺] or ferrocene complex is used. The phenanthroline cobalt (II) complex [Co(phen)₃²⁺] has advantages that an adjustment thereof can be easily, a reversible oxidation-reduction reaction can be performed, and an oxidation-reduction potential is relativity low and lowest in metal complexes having phenanthroline as a ligand. The phenanthroline cobalt (II) complex [Co(phen)₃²⁺] is added, for example, as a solution to the sample solution 5.

The mediator which is the most superior at this stage is the phenanthroline cobalt (II) complex. The phenanthroline cobalt (II) complex has an oxidation potential of about 200 mV lower than an oxidation potential of ferrocene. In this way, the phenanthroline cobalt (II) complex is possible to react in low potential, so the complex may avoid an influence of ascorbic acid or other substance.

The self-assembled monolayer 18 having the above chemical structural formula allows only substance causing the electrochemical reaction for measuring the object in the gold electrode 16 to pass through. Namely, the phenanthroline cobalt (II) complex or other mediator is allowed to pass through, and ascorbic acid, uric acid or other impurities in the sample solution 5 is not allowed to pass through and is repelled from the gold electrode 16. Ascorbic acid and uric acid are respectively included in a vegetable, a fruit, blood, or urine in which the measuring object is included relatively in large quantities, and causes the oxidation reaction in the gold electrode 16. The following formula indicates the oxidation-reduction reaction of ascorbic acid.

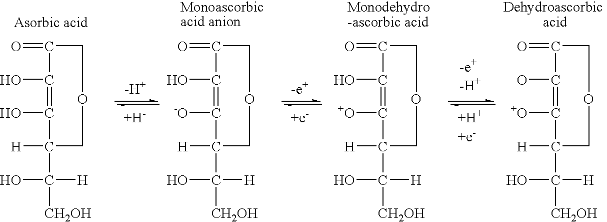

Ascorbic acid loses two protons in an oxidation reaction through monoascorbic acid anion and monodehydroascorbic acid to become dehydroascrobic acid. At that time, reactions concerning monodehydroascorbic acid generate a current.

In this way, if ascorbic acid exists near the gold electrode 16, the gold electrode 16 may be applied with an electrode reaction voltage to oxidize the ascorbic acid to thereby generate a current. Therefore, when the sample solution 5 includes ascorbic acid, the ascorbic acid operates as an interference substance which interferes in the electrode reaction of the measuring object and the gold electrode 16 to lower measurement accuracy of the concentration of the measuring object by using the sensor 10. Uric acid, similarly to the above, is oxidized to operate as the interference substance.

However, in the present embodiment, the self-assembled monolayer 18 repels ascorbic acid, uric acid, or other impurities to obstruct an arrival of the same to the gold electrode 16, and allows substance causing the electrochemical reaction for measuring the object to pass through selectively.

When measuring the concentration of the object in the sample solution 5 by using the sensor 10 having the above configuration, the potentio and galvanostat apparatus 7 accurately applies a predetermined voltage via the wire W4 to the reference electrode 13.

Further, the potentio and galvanostat apparatus 7 applies a predetermined voltage via the wires W1 and W2, which are connected to the modified electrode 12, to the modified electrode 12 by using the applied voltage to the reference electrode 13 as a reference. The voltage applied to the modified electrode 12, for example, is a voltage causing an electrode reaction of Co(phen)₃²⁺ and the gold electrode 16 described later.

By using the potential of the reference electrode 13 in which a value does not change due to no electrochemical reaction, the modified electrode 12 is applied with the voltage, consequently the potential of the modified electrode 12 can be retained and a fluctuation of the potential can be suppressed as the electrode reaction progresses.

In the sensor 10 according to an example of the present invention described above, the gold electrode 16 of the modified electrode 12 is modified by the self-assembled monolayer 18 and further immobilized with the enzyme 19. In this way, the arrival of the interference substance to the gold electrode 16, which causes reactions other than the electrochemical reaction for the measurement, can be prevented. As a result, selectivity of the modified electrode 12 is improved and the measurement accuracy of the measuring object by using the sensor 10 is improved.

The self-assembled monolayer 18 is formed by only dipping the gold electrode into a solution of thiol compounds, which allow a formation of the modified electrode 12 and the sensor 10 in low cost and simply.

Further, the apparatus and a measurement method of the present embodiment are not complicated such as oxidation-reduction titration or gas chromatography, the measuring object can be measured simply. And, having nothing to react glucose, a work for removing an influence of the glucose is unnecessary, so the concentration of the measuring object such as fructose can be measured simply and with high accuracy.

Further, in the present embodiment, the measurement is performed by three electrodes system in which three electrodes of the modified electrode 12, the counter electrode 11, and the reference electrode 13 are used and the electrode reaction voltage to the modified electrode 12 is applied by using the voltage applied to the reference electrode 13 as a reference. Therefore, the fluctuations of the potential of the modified electrode 12 due to the electrode reaction are suppressed, which achieves a high accuracy measurement.

(Embodiment of Fructose Sensor)

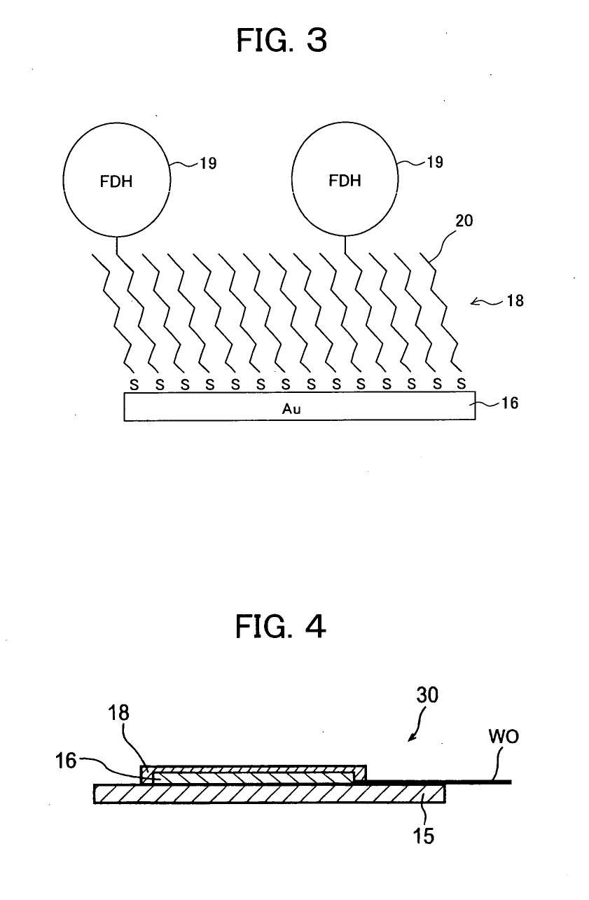

First, an embodiment of a fructose sensor for measuring concentration of fructose as the measuring object will be described. In the present embodiment, by using the self-assembled monolayer 18 as shown in FIG. 3, FDH is immobilized as the enzyme 19 to the gold electrode 16.

In the present embodiment, for example, the self-assembled monolayer 18 is formed of 7-carboxy-1-heptanthiol (7C). The 7C has the Au—S bonding and additionally Van der Waals interaction between the alkyl chains 20, which allows a formation of a monolayer having a high orientation and free from defects. In the present embodiment, FDH bonds an alkyl chain 20 of the self-assembled monolayer 18 made of 7C. Therefore, FDH is immobilized to the gold electrode 16.

FDH is pyrroloquinoline quinine (PQQ) oxidoreductase of a layer bond having a molecular weight of 140,000 Da of three subunits. FDH bonds PQQ strongly. FDH oxidizes D-fructose to form 5-keto-fructose, and PQQ is reduced to become PQQH₂.

In the present embodiment, the oxidation reaction of fructose by FDH is mediated by using a mediator of Co(phen)₃²⁺, and then converted to an electric signal in the gold electrode 16. The detection situation of fructose will describe in the following.

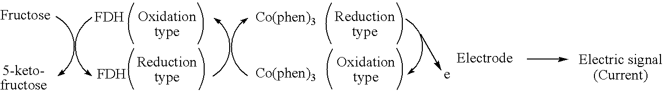

FDH oxidizes fructose in the sample solution 5 to generate a reduction type phenanthroline cobalt (II) complex. The reduction type phenanthroline cobalt (II) complex passes to the self-assembled monolayer 18, and then is oxidized at a surface of the gold electrode to generate an electric signal in the gold electrode 16.

Also, if the sample solution 5 in the vessel 3 includes ascorbic acid, uric acid, or other impurities, the impurities may be obstructed by the self-assembled monolayer 18 not to achieve the arrival to the gold electrode 16.

As described above, the self-assembled monolayer 18 of 7C repels ascorbic acid, uric acid, or other interference substance soluble in water and activated in electrochemically due to carboxyl group included in 7C, and obstructs the arrival to the gold electrode 16.

Below, an embodiment for verifying that the self-assembled monolayer 18 made of 7C has a selectivity to repel ascorbic acid will be described.

Ascorbic acid is oxidized at the surface of the gold electrode 16 to interfere in the electrode reaction in the gold electrode 16. So, in testing the influence of ascorbic acid in the electrochemical reaction for the measurement of fructose, FDH for oxidizing fructose is unnecessary. Therefore, by using the modified electrode being modified with only the self-assembled monolayer 18 made of 7C, the electrochemical reaction due to ascorbic acid is measured.

FIG. 4 is a cross-sectional view of the modified electrode used to the present embodiment. The modified electrode 30 shown in FIG. 4 is a modified electrode shown in FIG. 2B without the enzyme 19. Therefore, the same components are assigned the same notations and their descriptions are omitted.

The modified electrode 30 shown in FIG. 4, similarly to the modified electrode 12, is directly modified with 7C at the gold electrode 16 by the Au—S bonding to form the self-assembled monolayer 18.

The three electrodes system sensor 10 shown in FIG. 1 measured cyclic voltammetry of ascorbic acid for testing a relationship between an applied voltage and the electrode reaction by using the modified electrode 30 described above instead of the modified electrode 12.

In the vessel 3, there was ascorbic acid solution of 0.6 mM (M=mol/l) as the sample solution 5. The sample solution 5 was injected with nitrogen gas continuously.

Under the above condition, the voltage applied to the modified electrode 30 was increased at a sweep rate of 10 mV/sec, then decreased and swept, and a current flowing between the modified electrode 30 and the counter electrode 11 are detected to achieve a cyclic voltammogram.

As comparisons with this, the modified electrodes modified with the gold electrode 16 by FDH, glutamate, and (N-5-amino-1-carboxypentyl) imino diacetic acid (AB-NTA) were formed respectively, and the CV measurement thereof, similarly to the modified electrode modified with 7C, were performed.

The FDH modified electrode was formed by modifying 0.5 mg/ml of cystamine and 5% of glutaraldehyde to the gold electrode 16, introducing an aldehyde group, and then immobilizing 0.5 mg/ml of FDH. The glutamate modified electrode and the AB-NTA modified electrode were formed by, similarly to the above FDH modification, immobilizing glutaraldehyde, and then modifying only glutamate or AB-NTA respectively. Note that, glutamate molecular has no alkyl chain and two carboxyl groups. And, AB-NTA has three carboxyl groups in every single alkyl chain.

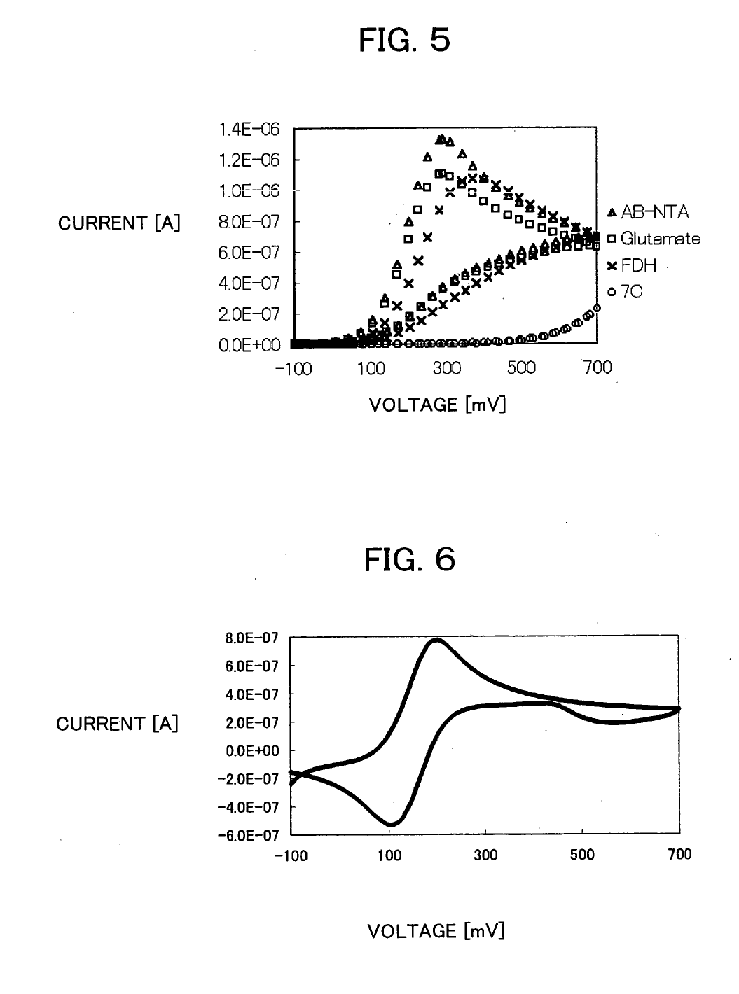

FIG. 5 shows the cyclic voltammograms for the above four modified electrodes.

In FIG. 5, abscissa indicates a voltage (mV) applied to the modified electrode, and ordinate indicates a current (A) detected by the potentio and galvanostat apparatus 7.

Further, “Δ” indicates a result in the case of using the AB-NTA modified electrode, “□” indicates a result in the case of using the glutamate modified electrode, “x” indicates a result in the case of using the FDH modified electrode, and “∘” indicates in the case of using the 7C modified electrode.

The voltage was increased, then decreased and swept, so that two current values are plotted in a single voltage.

FIG. 5 shows that the larger the current value is, the more the oxidation reaction of ascorbic acid is caused in the gold electrode 16.

As shown in FIG. 5, if assuming the result of the FDH modified electrode as a reference, a peak current may hardly change or become larger in the case of using the glutamate modified electrode and the AB-NTA modified electrode. The results show that the modifications by glutamate and AB-NTA are not able to achieve a positive effect for a prevention of the oxidation reaction of ascorbic acid.

On the other hand, in the case of using the 7C modified electrode, it is obviously from FIG. 5, the oxidation reaction of ascorbic acid was hardly observed. The result shows that the 7C modified electrode is greatly effective for the prevention of the oxidation reaction of ascorbic acid.

The above test is proved that the 7C modified electrode is effective for the prevention of the oxidation reaction of ascorbic acid. However, for using the 7C modified electrode as a fructose sensor, the 7C modified electrode is necessary to cause the electrode reaction with the mediator. For verifying this, Co(phen)₃²⁺ solution of 1 mM was added into the vessel 3 as the mediator, and then the CV measurement was performed by the sensor 10 using the 7C modified electrode. FIG. 6 shows the result.

Similarly to FIG. 5, abscissa of the cyclic voltammogram shown in FIG. 6 indicates the voltage (mV) applied to the modified electrode, and ordinate thereof indicates the current (A) detected by the potentio and galvanostat apparatus 7.

As shown in FIG. 6, a peak of an oxidation current is observed in about 200 (mV) and a peak of a reduction current is observed in about 100 (mV). From the above, it can be verified that the 7C modified electrode causes the oxidation-reduction reaction of Co(phen)₃²⁺.

The CV measurements of Co(phen)₃²⁺ described above were performed a plurality of times by fluctuating the concentration of 7C within 0.05 to 0.2 mg/ml and by using the 7C modified electrode having the self-assembled monolayer 18 respectively. As a result, at the concentration of 7C of 0.15 mg/ml, the peak value of the oxidation current of Co(phen)₃²⁺ was relatively higher and about 1257 times in comparison with that of ascorbic acid at the same voltage, so the interference of ascorbic acid in an oxidation reaction of Co(phen)₃²⁺ was minimum.

From this result, it is obvious that the self-assembled monolayer 18 using 7C allows Co(phen)₃²⁺ to pass through selectivity and the 7C modified electrode has a selectively for a measurement of fructose.

It was performed with a verification examination whether the 7C modified electrode functioned as fructose sensor in actually. An electrode having the modified concentration of 0.15 mg/ml was used and FDH were dissolved in Co(phen)₃²⁺ solution to be FDH concentration of 3 mg/ml, and the CV measurement was performed. Then, fructose was dropped to be 1 mM, and the CV measurement was performed.

As a result, in comparison with the case without an addition of fructose, the oxidation current value increased about 1.23 times in the case of the addition of fructose. Namely, the oxidation reaction of fructose due to FDH was measured as the oxidation current in the gold electrode 16 by Co(phen)₃²⁺.

As described above, the sensor having the 7C modified electrode achieves a practical application as the fructose sensor. The fructose sensor having the 7C modified electrode can suppress the interference of ascorbic acid, so it has high selectivity to fructose and measurement accuracy higher than the past.

(Embodiment of Glucose Sensor)

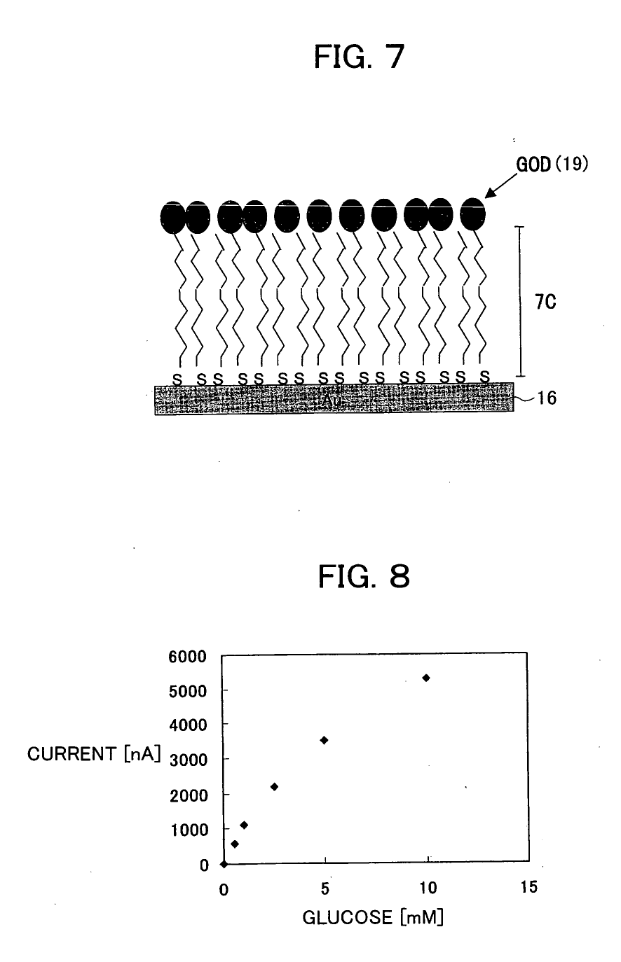

FIG. 7 is a view of a configuration of the glucose sensor. The glucose sensor was formed by immobilizing glucose oxidase (GOD) of an oxidase for glucose as the enzyme 19 on the self-assembled monolayer 18 made of 7C.

The immobilization of GOD may be performed by bonding directly 7C by a calbodiimide method used carboxyl group, or by providing a separated layer of GOD not bonding 7C. The measurement of glucose is performed by using Co(phen)₃²⁺, Fe(phen)₃²⁺ or other mediators which allows a charge transition with GOD passing through the self-assembled monolayer 18 made of 7C, but it is not limited to the above.

FIG. 8 shows a measured result in the case of a measurement using Co(phen)₃²⁺ as the mediator. As shown in FIG. 8, the detected current depends on the glucose concentration, so it can be used to a selective measurement of glucose. Note that, the point that the 7C modified electrode prevents the oxidation reaction of ascorbic acid at the gold electrode 16 was verified by the test shown in FIG. 5, and the point that the 7C modified electrode causes the electrode reaction with the mediator of Co(phen)₃²⁺ was verified by the test shown in FIG. 6.

As described above, it is obvious that the sensor having the 7C modified electrode achieves a practical application as the glucose sensor. The glucose sensor having the 7C modified electrode can suppress the interference of ascorbic acid, so it has a high selectivity to glucose and the measurement accuracy higher than the past.

(Embodiment of Catecholamine Sensor)

Embodiment applying the modified electrode modified by the self-assembled monolayer 18 to a measurement of dopamine which is one of catecholamine and which is neurotransmitter will be described.

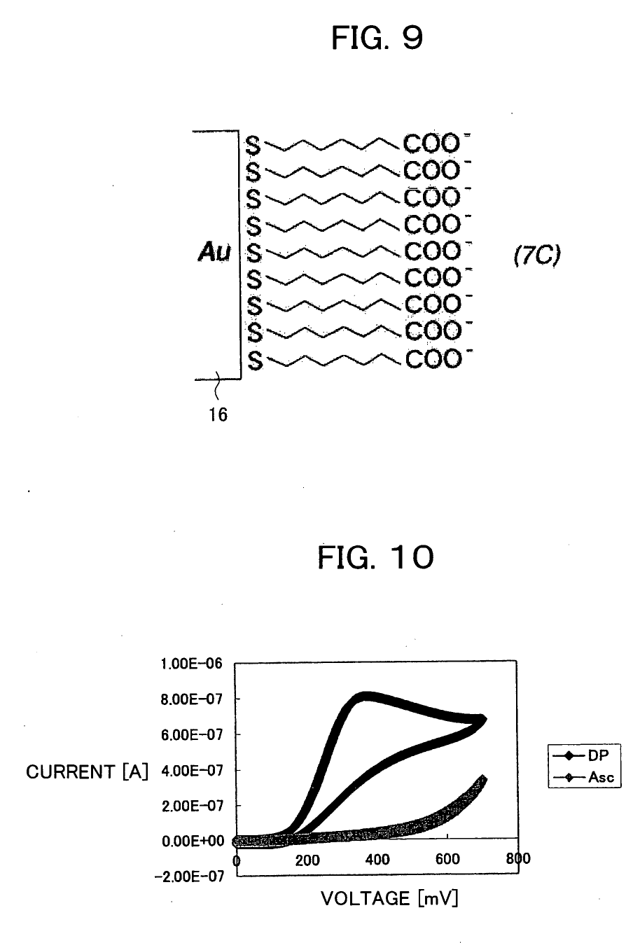

FIG. 9 is a view of a configuration of a catecholamine sensor.

The gold electrode 16 was dipped into 7C of 0.15 mg/ml to form the modified electrode shown in FIG. 9. The modified electrode was applied by the potentio and galvanostat apparatus 7 with a predetermined voltage to cause the electrode reaction.

FIG. 10 shows a result of the CV measurement of measuring a reaction of dopamine (DP) and ascorbic acid (ASc) by using the modified electrode. Abscissa of the cyclic voltammogram shown in FIG. 10 indicates a voltage (mV) applied to the modified electrode, and ordinate thereof indicates a current (A) detected by the potentio and galvanostat apparatus 7.

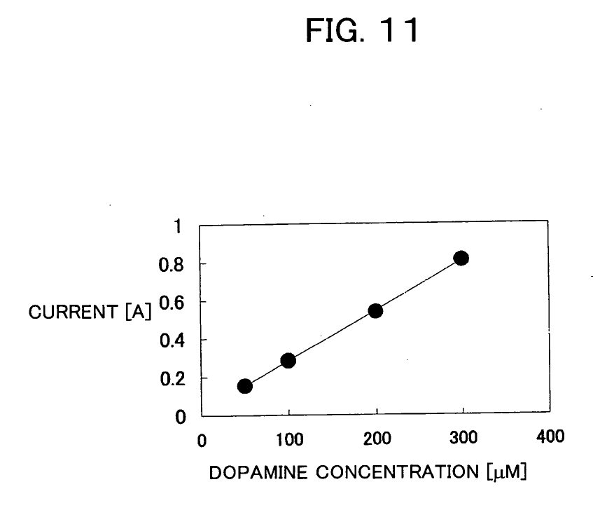

It is found from FIG. 10, a reaction similarly to the unmodified electrode was observed in dopamine, but the reaction was hardly observed in ascorbic acid. FIG. 11 shows a calibration curve of dopamine by using the modified electrode modified by the self-assembled monolayer. As shown in FIG. 11, the current of dopamine depends on a dopamine concentration, and can be used to the selectivity measurement of dopamine.

As described above, the sensor having the 7C modified electrode achieves a practical application as a sensor for catecholamine including dopamine. The catecholamine sensor having the 7C modified electrode can suppress the interference of ascorbic acid, so it has a high selectivity to catecholamine and measurement accuracy higher than the past.

(Embodiment of Glucose Sensor Made of HS(CH₂)ₙCOOH (n=5 to 9))

In the present embodiment, the self-assembled monolayer 18 is formed on the gold electrode 16 by 5-carboxy-1-pentanetiol (5C), 7-carboxy-1-heptanetiol (7C), and 10-carboxy-1-decanetiol (10C) which have different alkyl chain lengths respectively. In the respective layers, a ratio of a current of dopamine to the peak current of ascorbic acid (dopamine/ascorbic acid) was compared.

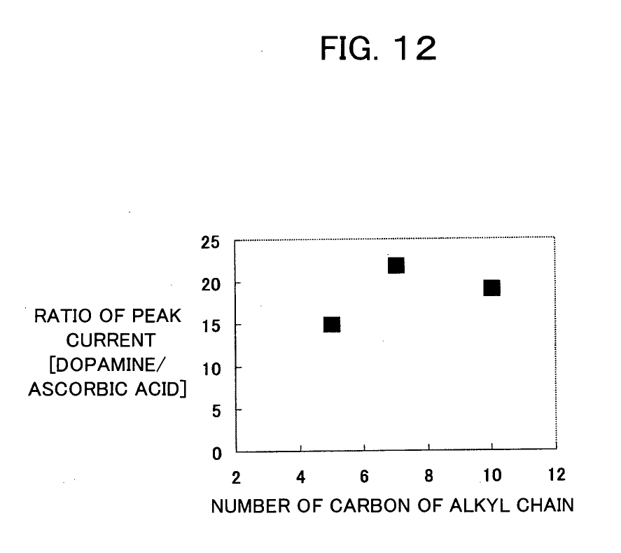

FIG. 12 is a view showing a comparison between an alkyl chain length of lipid included in the self-assembled monolayer and dopamine selectivity.

FIG. 12 indicates that 7C has a superior selectivity for dopamine. In this measurement, 5C also has the selectivity for dopamine, but is inferior to 7C. Note that, from a result shown in FIG. 12, it is easily estimated that 6-carboxy-1-hexanetiol (6C) having the alkyl chain between 5C and 7C, and 8-carboxy-1-octanetiol (8C) and 9-carboxy-1-nonanetiol (9C) having the alkyl chain between 7C and 10C have the selectivity for dopamine.

Further, although this embodiment shows that 7C is the most superior, if the measuring object is different from above, 5C, 6C, 8C, or 9C may have, similarly to 7C, a superior selectivity for the measuring object.

(Modification of Apparatus Configuration)

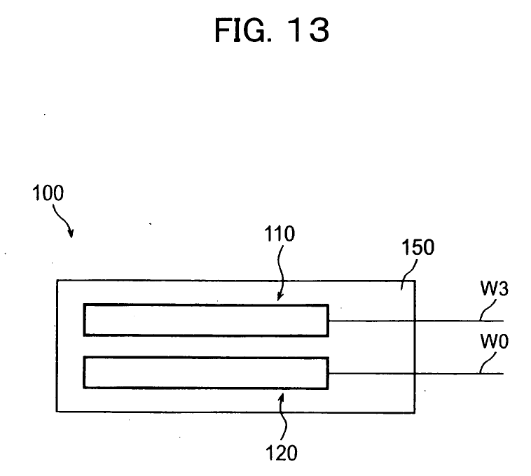

FIG. 13 is a plane view of a sensor module according to a modification of an embodiment of the present invention.

A sensor module 100 shown in FIG. 13 is integrally provided with a modified electrode 120 and a counter electrode (CE) 110 opposed to a grass, plastic, or other substrate 150.

The modified electrode 120, similarly to the modified electrode 12 shown in FIG. 2, is formed on a surface of the gold electrode with the self-assembled monolayer, and immobilized with the enzyme. The modified electrode 120 is connected via the wire W0 to the potentio and galvanostat 7, and the counter electrode 110 is connected via the wire W3 to the potentio and galvanostat 7.

By the above configuration, the modified electrode 12 and the counter electrode 11 shown in FIG. 1 are modularized to produce the sensor. According to the present modification, the electrodes are modularized, which allows a formation of the sensor in small size with easily handling.

The embodiments of the present invention were described, and the present invention is not limited to the above embodiments.

For example, the self-assembled monolayer 18 according to the present embodiments can repel uric acid or other substance soluble in water and activated in electrochemically other than ascorbic acid, and keep the same at a distance from the electric board.

The sensor according to the present invention is applicable to a fructose sensor, a glucose sensor, a catecholamine sensor, additionally various types of biosensors or chemical sensors.

If using the measurement object allowing the self-assembled monolayer 18 to pass through directly, the enzyme 19, mediator, or other component may be unnecessary to use.

Further, if a sensor having two electrodes system free from the reference electrode 13 is produced, it may be impossible to achieve the measurement accuracy of the three electrodes system. Due to the two electrodes system sensor, the sensor can be made smaller in size and lower in cost.

Note that, the counter electrode used to the sensor can use platinum mesh, platinum plate, or other various electrodes. Also, the electrode base modified by the self-assembled monolayer can use other metal other than gold, and can be made plate-shaped, line-shaped, or other various shaped. Note that, as the electrode base for the sensor, gold is the most superior.

As described above, according to the present invention, the electrode able to suppress an influence of the interference substance in measuring the object by using the self-assembled monolayer and improvable to the measurement accuracy, and the sensor using the same can be provided.

### INDUSTRIAL APPLICABILITY

The electrode according to the present invention and the sensor using the same, depending on the measuring object, can be applied to food engineering, clinical, industry, chemistry, or other various fields.

1. An electrode comprising:
an electrode base, and
a self-assembled monolayer expressed by a chemical structural formula of HS(CH₂)ₙCOOH (n=5 to 9) and covering said electrode base.

2. An electrode as set forth in claim 1, wherein an enzyme is immobilized on said self-assembled monolayer, said enzyme making an object to be measured oxidation-reduction react.

3. A sensor comprising:
a vessel receiving a sample solution in which an object to be measured dissolves, and
a modified electrode and a counter electrode to be dipped into said sample solution,
wherein said modified electrode comprises
an electrode base, and
a self-assembled monolayer expressed by a chemical structural formula of HS(CH₂)ₙCOOH (n=5 to 9) and covering said electrode base.

4. A sensor as set forth in claim 3, wherein a mediator is added into said sample solution, said mediator transferring a charge with said electrode base under said oxidation-reduction reaction of said object.

5. A sensor as set forth in claim 4, wherein a hydrophobic mediator is added into said sample solution.

6. A sensor as set forth in any one of claims 3 to 5, wherein an enzyme is immobilized on said self-assembled monolayer, said enzyme making said object oxidation-reduction react.

7. A sensor as set forth in claim 3, further comprising:
a voltage applying means for applying an electrode reaction voltage to said modified electrode, and
a calculation means for calculating concentration of said object based on an electric current flowing between said modified electrode and said counter electrode.

8. A sensor as set forth in claim 7, further comprising a reference electrode,
wherein said voltage applying means applies a predetermined voltage on the basis of a voltage of said reference electrode to said modified electrode.
